(19) United States
(12) Patent Application Publication
(10) Pub. No.: US 2007/0009089 A1
(43) Pub. Date: Jan. 11, 2007

(54) Methods and apparatuses for making x-rays using electron-beam ion trap (EBIT) technology

(75) Inventor: Marcus H. Mendenhall, Nashville, TN (US)

Correspondence Address: FULBRIGHT & JAWORSKI L.L.P., 600 CONGRESS AVE., SUITE 2400, AUSTIN, TX, 78701, US

(73) Assignee: Vanderbilt University

(21) Appl. No.: 11/175,409

(22) Filed: Jul. 6, 2005

(51) Int. Cl. A61N 5/10

(52) U.S. Cl. 378/65

(57) ABSTRACT

Methods and systems for making X-rays using Electron-Beam Ion Trap (EBIT) technology. A method includes extracting ions of various atomic species from an EBIT, transporting the ions through an evacuated tube, and producing x-rays by neutralizing the ions when the ions strike a conducting plate. Another method includes producing x-rays through EBIT technology and using the x-rays in a medical application. An apparatus includes an EBIT configured to emit fully ions of various atomic species, an evacuated tube configured to transport the ions, and a conducting plate configured to produce X-rays by neutralizing the ions when the ions strike the conducting plate.

This application claims priority to, and incorporates by reference, U.S. Provisional Patent Application Ser. No. 60/575,305 entitled “METHODS AND APPARATUSES FOR MAKING X-RAYS USING ELECTRON-BEAM ION TRAP (EBIT) TECHNOLOGY,” which was filed on Jul. 6, 2004.

### STATEMENT AS TO RIGHTS TO INVENTIONS MADE UNDER FEDERALLY-SPONSORED RESEARCH AND DEVELOPMENT

Aspects of this invention were made with government support pursuant to grant number FA9550-04-1-0045 from the Department of Defense Medical Free Electron Laser (DOD MFEL) Program. Accordingly, the government may have certain rights in this invention.

### BACKGROUND OF THE INVENTION

1. Field of the Invention

The present invention relates generally to techniques for making x-rays. More particularly, it concerns using Electron-Beam Ion Trap (EBIT) technology to generate x-rays that can be used for medical applications such as, but not limited to, radiation oncology and activation of drugs for cancer therapy.

2. Description of Related Art

Presently, there are drugs containing a heavy element such as iodine, gadolinium, or platinum, that can be used as radiation sensitizer agents for tumor therapy. The drugs generally deposit large amounts of energy in a small volume via Auger electron emission and have a high probability of creating double-stranded breaks on DNA if the decay occurs in the cell nucleus, and thus, causing cell lethality. One option for providing the requisite excitation in an atom to create an Auger cascade is radioactive decay of the atom. For example, one class of radioactive species commonly used is the radioactive thymidine analogs such as iododeoxyuridines (IUdRs), (e.g., [₁₂₃I]UdR and [₁₂₅I]UdR). The difficulty with using radioactive species for the purpose of curative treatment of cancer is that unless the species have very high tumor specificity, they can cause damage throughout the body, e.g., cause damage to normal tissues. Thus, great effort must be made to confine the drug to the region of interest.

Another method for providing the excitation in an atom to generate auger electrons is by an independent source of ionization, which has the potential advantage to achieve a strong synergistic cell killing. The in dependent source, in combination with a radiation sensitizing agent, may result in very high damage. In particular, one example of a radiation sensitizing agent is IUdR. IUdR is a low-toxicity, tumor-avid drug which when combined with an applied radiation, is enhanced in the region where the drug is localized. Another example is the cis-diamminedichloroplatinum (II) (cDDP) drug, which has moderately high toxicity alone, but may achieve extra impact when activated with ionizing radiation.

Unfortunately, sources used for ionization, such as x-rays, are currently limited and expensive. Conventional x-rays sources, such as x-rays tubes and accelerators, produce such a broad spectrum of photons that any enhancement due to the strong absorption near an elemental K-edge is washed out. Narrow-band sources such as synchrotrons are well established but are expensive (around $1 billion) for routine work. Compton-backscattering sources, which are less expensive, are too complex and experimental.

Referenced shortcomings of conventional methodologies mentioned above are not intended to be exhaustive, but rather are among several that tend to impair the effectiveness of previously known techniques concerning a source for generating x-rays for radiation oncology. Other noteworthy problems may also exist; however, those mentioned here are sufficient to demonstrate that methodologies appearing in the art have not been altogether satisfactory and that a significant need exists for the techniques described and claimed here.

### SUMMARY OF THE INVENTION

Certain shortcomings of the prior art are reduced or eliminated by the techniques disclosed here. These techniques are applicable to a vast number of applications, including applications in medical radiotherapy.

In one respect, the disclosure involves a method for using x-rays in a medical application. The method includes extracting ions an Electron-Beam Ion Trap (EBIT). The ions may be stripped ions. Alternatively, the ions may be hydrogenic ions. The ions are transported through an evacuated tube to a conducting plate. When the ions strike the conducting plate, the ions become neutralize (electrically neutral), and produces x-rays. The x-rays may subsequently be delivered to a patient.

In another respect, the disclosure involves a method for irradiating a tumor in a patient. The tumor may be provided a radiation sensitizer agent. Next, x-rays are delivered to the patient. The x-rays may be produced from extracting ions from an EBIT, transporting the ions through an evacuated tube, and neutralizing the ions when the ions strike a conducting plate. The radiation sensitizer agent is activated by the x-rays, and the tumor is irradiated.

In other respects, a system is provided. The system includes an Electron-Beam Ion Trap (EBIT), an evacuated tube, a conducting plate, and a filter. The EBIT is configured to emit ions, such as stripped ions or hydrogenic ions. The evacuated tube is configured to transport the ions from the EBIT to the conducting plate. When the ions strike the conducting plate, the ions become neutralized and produce x-rays. The filter is configured to filter l-series energy photons from the x-rays. The x-rays can be used at a variety of discrete energies for medical applications.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus or method that “comprises,” “has,” or “includes” one or more elements or steps possesses those one or more elements or steps, but is not limited to possessing only those one or more elements or steps. Likewise, an element of an apparatus, or a step of a method, that “comprises,” “has,” or “includes” one or more features or steps, possesses those one or more features or steps, but is not limited to possessing only those one or more features or steps.

The terms “a” and “an” are defined as one or more than one unless this disclosure explicitly requires otherwise.

Other features and associated advantages will become apparent with reference to the following detailed description of specific embodiments. Along with this disclosure, the claims of this application take into account the breadth of the present invention.

### BRIEF DESCRIPTION OF THE DRAWINGS

The following drawings form part of the present specification and are included to further demonstrate certain aspects of the present invention. The figures are examples only. They do not limit the scope of the invention.

FIG. 1 is an illustration of an electron beam ion trap.

FIG. 2 is a graph illustrating x-ray signals as a function of an axial trapping voltage, in accordance with embodiments of this disclosure.

FIG. 3 is a graph illustrating absorption cross sections at K-edges for various elements, in accordance with embodiments of this disclosure.

FIG. 4 shows models of iodine-loaded tumors treated at different energy levels, in accordance with embodiments of this disclosure.

FIG. 5 shows models of platinum-loaded tumors treated at different energy levels, in accordance with embodiments of this disclosure.

FIG. 6 is a model of an iodine-loaded tumor treated with a beam delivered to the center of the tumor, in accordance with embodiments of this disclosure.

FIG. 7 illustrates a method for delivering beams to a patient, in accordance with embodiments of this disclosure.

FIG. 8 illustrates a method for delivering beams to a patient, in accordance with embodiments of this disclosure.

### DESCRIPTION OF ILLUSTRATIVE EMBODIMENTS

The description below is directed to specific embodiments, which serve as examples only. Description of these particular examples should not be imported into the claims as extra limitations because the claims themselves define the legal scope of the invention. With the benefit of the present disclosure, those having ordinary skill in the art will comprehend that techniques claimed and described here may be modified and applied to a number of additional, different applications, achieving the same or a similar result. The attached claims cover all such modifications that fall within the scope and spirit of this disclosure.

Substantially monochromatic x-ray photons may be produced using fully stripped or hydrogenic (single-electrons) heavy ions in an Electron-Beam Ion Trap (EBIT) source. The stripped ions produced may be transported as a beam and may be manipulated with magnetic and electrostatic optics to steer and focus the beam onto its final target. In one embodiment, the target may be a plate or collimator that would produce an x-ray beam directed at the outside of a patient. Alternatively, in other embodiments, the target may be a hollow cannula inserted into a tumor, allowing exposure from within a patient.

EBIT Technology

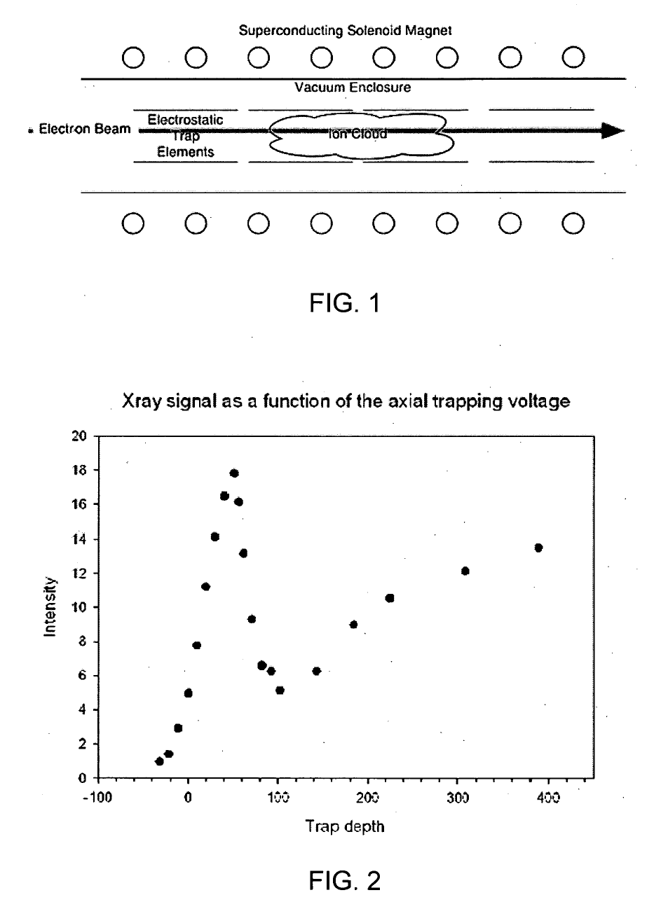

The basic design of an EBIT involves a high-energy electron beam having low-energy ions passing through electrostatic trap elements and an ion cloud in a combined magnetic and electrostatic field, as shown in FIG. 1. The ion cloud may include energetic electrons which may strip the low-energy ions of the electron beam via impact ionization. For example, current EBIT devices are capable of stripping substantially all of the electrons from naturally occurring elements such as a uranium (creating U⁹²⁺). Details of the stripping process are discussed further below.

To make an EBIT practical for producing high extracted currents, the EBIT device may operate with a high electron beam current to recapture the electron beam energy in a depressed collector. This may allow for both the beam power requirements and radiation shielding for the beam stop to be minimized. Typical beam energies used are up to approximately 200 keV at currents of up to approximately 5 A. At this energy and current level, beam power may correspond to approximately 1000 kW, which would require substantial shielding and cooling of the electron beam dump. In contrast, using a depressed collector, the beam may be recaptured at a potential difference of about 1 kV or less from the potential of the source, resulting in the electrons stopped having an energy of about 1 keV and electrical power and cooling of below 5kW. At this energy level, soft x-rays may be produced and may easily be shielded.

During the basic operation of the EBIT, a high current electron beam may be launched from an electron gun and may get compressed by a high magnetic field in the central part of the EBIT device. Ions may be created by direct and resonant electron impact ionization processes in the drift tube trap region. In some embodiments, voltages on three cylindrical drift tubes may create an axial trapping potential well for the ions. In the radial direction, the trapping may be done by the electrostatic attraction by the electron beam itself. As the ions are trapped longer, their charge state may become increasingly higher until a balance develops between the ionization, recombination, and loss processes. The equilibrium charge state distribution may depend on the energy of the electron beam, but the dynamics of the ion confinement may be crucial in the ultimate rate of high charge state ion production in the machine.

In an EBIT device, the dynamics of the ion cloud may affect the charge states of the ions in the device. In some embodiments, ions may be situated in the V(r) potential well created by the electron beam as determined by the Boltzmann formula: n(r)=n₀ exp(−qV (r)/kT). Cooler ion clouds with the same number of ions can have a larger overlap integral with the electron beam and therefore, the ions can be stripped to higher charge states before leaving the trap. The formula also implies that lower charge ions may be less deeply bound and can leave the trap more easily. As such, by injecting a lower atomic number coolant gas into the EBIT, the accumulation of heavier and higher charged ions via the evaporative cooling mechanism may be enhanced. The same cooling principle may be used in the research of neutral atomic species to create Bose-Einstein condensates at very low temperatures. The removal of only a few percent of the higher energy particles can reduce the cloud temperature by orders of magnitudes. In the EBIT device, for every ion, there may be an ideal coolant gas that has a high enough charge and mass to remove energy efficiently from the trap but low enough to get deeply trapped and force the heavier ions out of the trap.

Both the ionization and the trapping may be determined by the electron beam screened by the ions and it may be possible that there are feedback mechanisms that can enhance or destroy the ideal trapping conditions. These effects have not been immediately recognized by the EBIT community; however, recent findings at the NIST EBIT indicate that they play important roles in the operation of the machine. For example, FIG. 2 provides an illustration for such an unexpected enhancement. As the axial trapping voltage is decreased from the maximum of about 500 V, the x-ray signal (proportional to the overlap integral between the electron beam and the ion cloud) slowly drops. However, at the region where the trapping changes over from radial to axial dominated, there may be a strong increase of the observed signal corresponding to a large enhancement in the number of ions. The phenomena needs to be further investigated and can give rise to new enhanced modes of operation of the EBIT.

Over the past couple of years there has been an enormous progress in the field of non-neutral plasmas. In these experiments, ions that are trapped in Penning traps can be cooled to low temperatures by different techniques. It has been shown that at high densities and low temperatures the ion clouds can go through phase transitions. At very low temperatures, the ions can form symmetric crystalline like structures. There is a dimensionless parameter Γ, known as the coulomb coupling parameter, that determines the correlation between the particles and is indicative of where a phase transitions take place. Γ is also the ratio between the potential energy due to the nearest neighbor of an ion to its kinetic energy and can be defined as follows:

$$Γ = \frac{{q}^{2}}{4 {πɛ}_{0} akT} \qquad Eq . 1$$

where a³=¾πρ and ρ is the density of the ion cloud. For ions at around Γ=173, the cloud forms a crystalline lattice. When Γ=1, the ions show a liquid like behavior.

EBIT ions are generally considered to be at a fairly high temperature. Since the density of the cloud can be very high due to the presence of the electron beam and since the ions are in high charge states, it may be possible that EBIT ions can go through a phase transition and collapse to the high electron density regions. In one embodiment, the coulomb coupling parameter may be used to induce a phase transition to enhance the EBIT operation. In other embodiments, in order to increase the density of the ion cloud in non-neutral plasmas the introduction of a rotation into the ion cloud may be used. Earlier attempts used lasers to induce rotation, but the rotations were uncontrollable. In one embodiment, the rotation of the ion cloud may be done using the wall technique, which provides a more stable and better controllable solution. The wall technique, as used herein, may include a rotating electric field that may be applied from electrodes near the wall of a vacuum system. The electric field may drag the ions in an orbit around the center of the chamber. As the ions rotate in the field, the Lorentz force compresses the cloud towards the axis of rotation. In this case, the center drift tube may be segmented and the voltages on the segments may vary due to the frequency of the rotation. As such, the ion cloud may be compressed due to the magnetic field.

Similar to the compression of the ion cloud, the electron beam density may also be determined by its rotational state. As the electrons enter the high magnetic field region of the EBIT, the collective rotation of the electrons induces the compression of the beam. The initial angular momentum of the electrons at the place of their emission may be a factor in forming the beam. Changes to the magnetic field at the place of the electron gun may enhance the emitted x-ray intensity and probably the charge state distribution of the ions.

One issue that arises in the application of an EBIT source is whether some of the stripped or hydrogenic ions (single-electron ions), when allowed to neutralize at a metal surface, lose their energy non-radiatively. In other words, the ions recaptures all the electrons stripped in the EBIT. The answer to this depends on the atomic number of the ion in question. For light ions, much of the de-excitation of core holes is via Auger decay. As one moves to heavier species, radiative de-excitation dominates. Even for ions as light as argon, the yield exceeds 20%. If one is working with species such as barium and bismuth, the conversion yield should be essentially 100%.

X-Ray Interactions with Radiation Sensitizer Agents

In order to maximize treatments of cancer (particularly irradiating tumors, and/or cancer cells) the combination of drugs, radiation sensitizer agent(s), and radiation source need to be tuned in a manner so that the most damage is inflicted. In one embodiment, the radiation source of the present disclosure may provide a wide range of x-ray photon energies such that one source may be used with substantially any combination of drug(s) and/or radiation sensitizer agent(s). A Monte-Carlo modeling program may be used to keep track and detail the interactions of an incident x-ray beam with a heterogeneous target. In one embodiment, the program may track the interactions by coherent (Rayleigh) and/or incoherent (Compton) scattering and photoelectric absorption. Any δ-rays produced by the interaction of the x-ray and target may be tracked until the rays lose most of their energy due to impact ionization of atoms in the material. The excited atoms, which may be produced by the x-rays or indirectly by the δ-rays may re-emit their energy either by Auger decay or fluorescent emission of another x-ray. Details of the modeling Monte-Carlo is discussed in more detail below.

It is noted that while generating detailed traces of the physics in tissue may be straightforward, the modeling program may not predict cell damage or death, since the mechanisms coupling energy deposition to these outcomes is poorly understood. As such, in one embodiment, the modeling program of the present disclosure may directly connect various energy deposition mechanisms to biological outcomes.

1. Scaling and Monte Carlo Modeling

Since the efficiency in killing or damaging cells may be a product of efficiency in absorbing photons by an element, i.e., the efficiency of an excitation of that element in killing or damaging cells, the probability may calculated as follows:

$$η = {\sum }_{Z , n} {f}_{n} ( Z ) \cdot σ ( Z , {E}_{0} {E}_{f} ) \cdot {Q}_{n} ( Z , {E}_{f} ) \qquad Eq . 1$$

where η is the probability of an incoming photon killing a cell, fₙ (Z) is the number density of atoms with atomic number Z at a site of type n in the cell, σ(Z, E₀E_{f}) is the cross section for an incoming photon of energy, E₀ is an atomic excitation of E_{f}, and Qₙ(Z, E_{f}) is the probability of an atomic species Z, excited at E_{f}, residing at site n, killing or damaging the cell. It is noted that the function a may include all indirect processes for exciting the atom, including, without limitation, fluorescence and subsequent recapture, ionization through a Compton-scattered electron, etc.

There may two mechanisms for primary excitation of an atom from an incoming x-ray. In one embodiment, photoelectric absorption or Compton scattering may be used. These two mechanisms may have different variations with incoming photon energy, and the combination of the two may affect the overall probability. For the purposes of scaling, the Thompson scattering cross section is as follows:

$$\frac{ⅆ σ}{ⅆ Ω} = {( \frac{{e}^{2}}{m {c}^{2}} )}^{2} \cdot 0.5 ( 1 + {cos}^{2} θ ) \qquad Eq . 2$$

where the energy transfer is

$${E}_{recoil} = {E}_{0} ( 1 - {( 1 + 2 \frac{{E}_{0}}{m {c}^{2}} {sin}^{2} \frac{θ}{2} )}^{- 1} ) \qquad Eq . 3$$

When the photon energy is below 100 keV, such that

$$2 \frac{{E}_{0}}{m {c}^{2}}$$

is approximately less than 0.4, the expansion of Eq. 3 is

$${E}_{recoil} \approx 2 \frac{{E}_{0}}{m {c}^{2}} {sin}^{2} \frac{θ}{2} \qquad Eq . 4$$

which, if the interest is in a fixed energy transfer Eₘᵢₙ to create an excitation sufficient for a double-stranded break (DSB) in DNA, gives a minimum scattering angle θₘᵢₙ of

$${sin}^{2} \frac{θ}{2} = \frac{1}{{E}_{0}} \sqrt{0.5 m {c}^{2} {E}_{min}} \qquad Eq . 5$$

As such, the maximum energy which may be transferred by Compton scatter is approximately

$${E}_{max} \approx 2 \frac{{E}_{0}}{m {c}^{2}} .$$

If this energy is insufficient to create a DSB, then Compton scattering isn't a contributor to useful damage. Furthermore, the total cross section for DSBS is approximately the integral of Eq. 2 from θₘᵢₙ to π such that θₘᵢₙ decreases with increasing E₀ and the Compton cross section increases with increasing beam energy, but may be non-selective to atomic species it excites and where damage can occur.

In some embodiments, the photoelectric components may be used in determining if a beam can cause DSB. The strength of the photoelectric absorption in heavy atoms may scale approximately with 1/E₀³ with discontinuities at the various x-ray edges. Since edges other than the K-edge are at very low energy, the K-edge may be only considered.

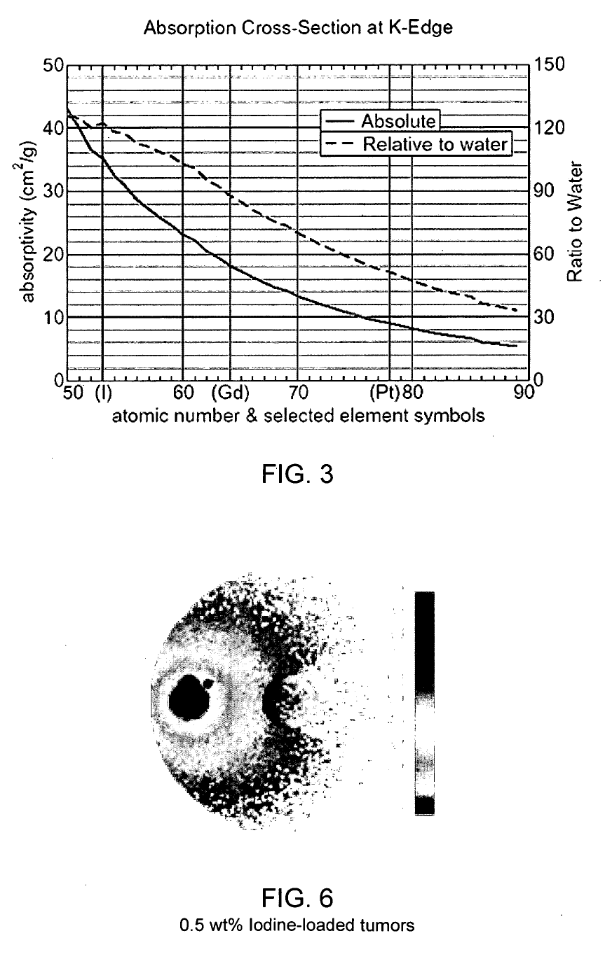

Referring to FIG. 3, the total x-ray scattering cross-section for every heavy element from tin (Z=50) to actinium (Z=89) for x-rays at an energy above the respective element's K-edge is shown. The graph illustrates the absolute absorptivity expressed in radiological units of cm²/g. As the heavier elements are considered, the absorption just above the K-edge gets progressively weaker. This is despite the increasing electron density of heavier elements. The graph also illustrates that as energy increases, the absorption by untreated tissue (mostly water) is also decreasing, as shown on the right-hand side of the graph of FIG. 3. This may be due to the selectivity being the ratio of the absorption strength of the heavy element at its K-edge to the absorption of water at the same energy. This measures how much more dose is provided to the heavy target than to the surrounding tissue. Thus, relatively light tags such as iodine show much higher selectivity than heavy tags such as platinum. However, platinate drugs are effective as well, and will be discussed in further details below. As such, due to benefits of both the light and heavy tags, the need for x-ray sources that may be used over an entire range of K-edge energies is apparent.

2. Monte-Carlo Calculations

The main goal of any calculation is to predict and/or determine cell damage and death from various system parameters, such as but not limited to, drug type, drug concentration, x-ray beam spectrum, and radiation dose. These calculations can optimize therapy such that effective treatment may be delivered to tumor cells and reduce exposure to non-cancer cells.

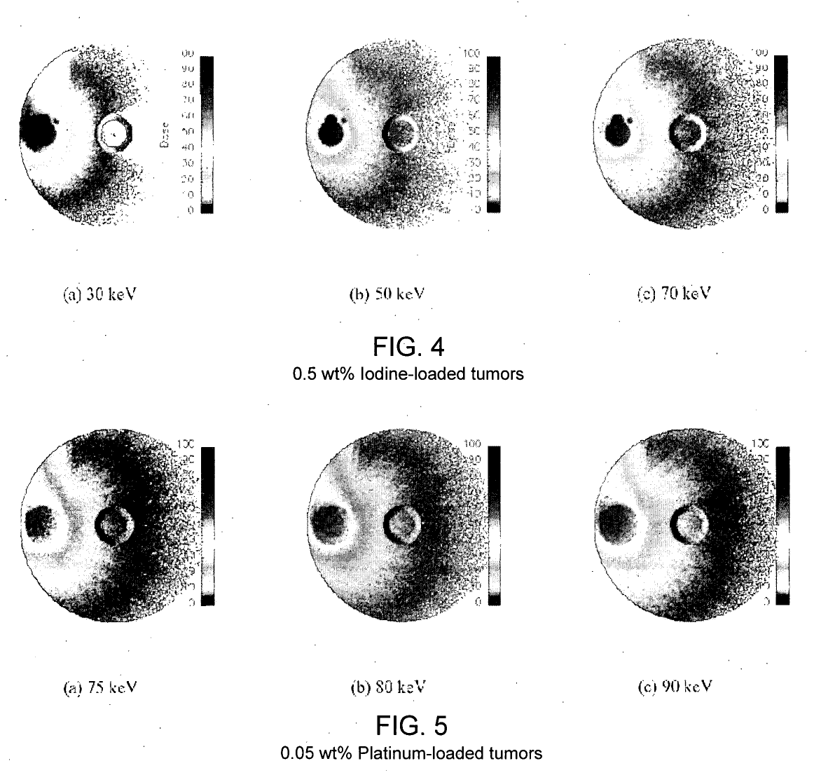

Referring to FIGS. 4 and 5, raw radiation exposure to energy deposited by Auger cascades is shown. FIG. 4 illustrates three energy levels used on iodine-loaded tumors while FIG. 5 illustrates 3 energy levels used on platinum-loaded tumors. The system includes a model tumor with the composition BR-12, a tissue-equivalent plastic simulating breast tissue. The ring centered in each image of FIGS. 4 and 5 models a bone made of a composition based on ICRU-44 cortical bone. The main body of the tumor is a cylinder with a diameter of two centimeters and the tumor has a small outlier to demonstrate the potential of the method of the present disclosure. The outlier may include fringes, imitating oddly shaped tumors.

In one embodiment, at the various different exposures, the beam or patient may be rotated around an axis perpendicular to the plane of the image. The rotation may be centered at the center of the main tumor body. As seen in FIGS. 4 and 5, the tumor is irradiated more effectively at the lower energy level.

In other embodiments, the beam may be delivered to the center of the tumor via a cannula, results modeled in FIG. 6. The tumor is an iodine-loaded tumor and is irradiated from the center. This technique allows the x-ray beams to emit into the full 4π solid angle from the center. By delivering a substantially entire beam to the center of the tumor, the effect to normal cells is reduced if not eliminated. Further, this method provides many of the characteristics of seed-based brachytherapy with the advantage of wide range selectable energies and the avoidance of highly-radioactive seeds.

A High-Current EBIT Device

In most current traps, the core structure is operated at cryogenic temperatures inside the bore of the superconducting magnet. Current trap designs provide a window to the gas cloud in the trap such that when the x-rays are generated from within the trap, magnets can draw the x-rays through the window. The window generally requires a split-winding magnet on both side of the window. This configuration requires extensive custom machining of parts and intricate integration with the structure of the magnet itself. In one embodiment of the present disclosure, a warm-bore magnet may be configured and designed such that the trap may be inserted. The warm-bore magnet may extract beam from the trap, and subsequent generation of x-ray beams may be done outside of the center of the trap. This configuration require little to no access to the center of the trap, thus simplifying the design of the device.

In one embodiment, the trap of the EBIT may be modified for achieving highest extracted current and increasing the operational voltage to allow stripping of bismuth. In addition, the EBIT may include a simple extraction beam line to permit ions to be brought to a target and to generate x-rays outside of the trap in different geometries, i.e., varying the x-rays for specific areas of the body and/or types of cells, etc. The EBIT may be constructed such that a flux from the trap of 10¹⁰ fully-stripped or hydrogenic particles per second may be achieved. This may allow the producing of the same number of x-rays in a 4¼ solid angle. At a distance of 1 cm from the target, the EBIT may provide a photon flux of approximately 109 cm⁻²s⁻¹. At 50 keV, which may be a suitable energy level for irradiating iodine-based drugs (where the dose rate may be in the range of approximately 0.04 Gray/second, e.g., a 10 Gy dose could be delivered in 250 seconds). Note that, because of the unusually effective cell killing associated with the drugs of interest, the required dose is likely to be much less than this.

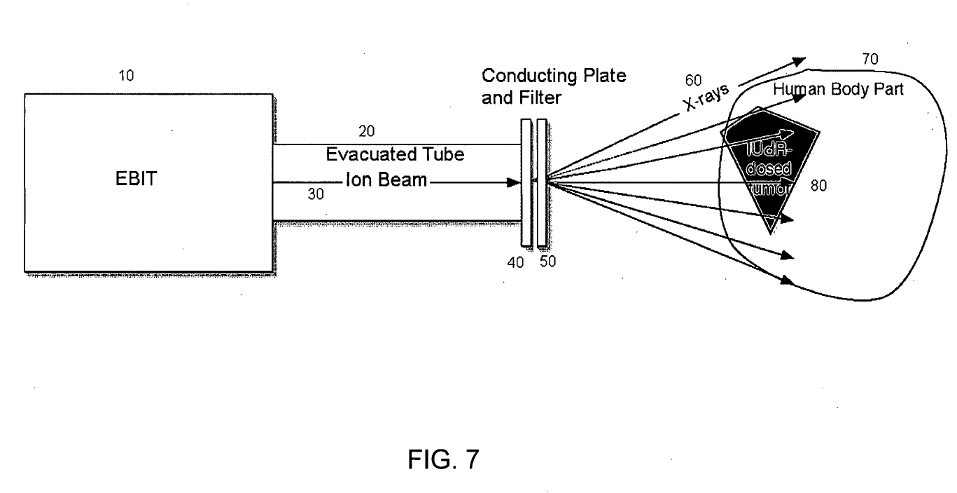

To create the x-rays, extracted beams from a trap may be directed to a target. For example, referring to FIG. 7, a system for generating x-rays from an extracted beam is shown. The system includes EBIT 10, evacuated tube 20, ion beam 30, conducting plate 40, filter 50, x-rays 60, human body part 70, and Iodinated Deoxyuridine (IUdR)-dosed tumor 80. In some embodiments, EBIT 10 may comprise an EBIT core unit, a high voltage power supply, a selectable ion source, a trapping magnet and an electron gun. Some embodiments may also comprise a selector for selecting a variety of ions, e.g., selecting an x-ray energy.

In some embodiments, fully stripped ions or hydrogenic ions of various atomic species may be extracted from EBIT 10, where the ions may be transported through evacuated tube 20. When the ions strike the conducting plate 40, they may become neutralized (e.g., recapturing all the electrons which were stripped off in the EBIT) and may produce x-rays. In some embodiments, the ions transported through the evacuated tube 20 may be transported as an Ion Beam, like Ion Beam 30. Ion beam 30 may be focusable in a straight line. Alternatively, ion beam 30 may be focusable in a non-linear path. In some embodiments the beam may be steerable, while in other embodiments, the beam may be both focusable and steerable. X-rays 60 may include essentially non continuous-spectrum x-rays. In some embodiments, x-rays 60 may comprise tunable x-rays. The x-rays may also comprise step-tunable x-rays or line x-rays at a variety of discrete energies with sufficient intensity for the medical application. This list is not by way of limitation. After the selection, filter 50 may be configured at a low energy to remove l-series energy photons.

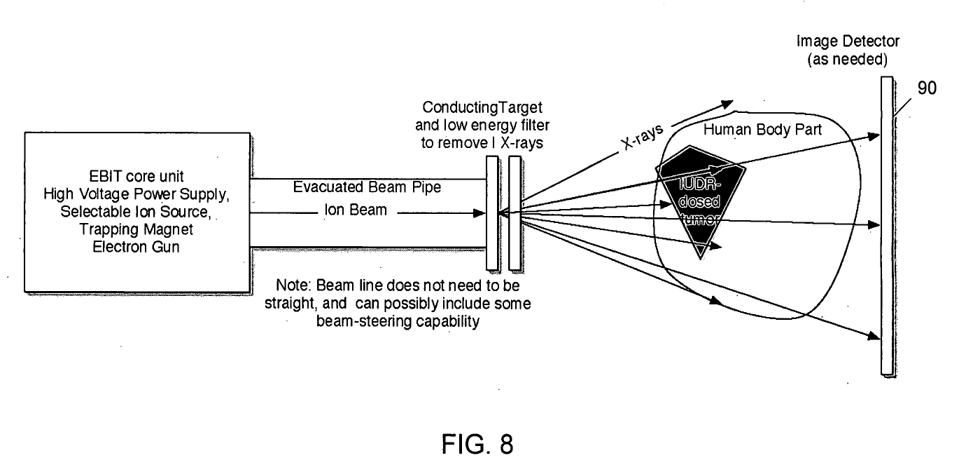

In some embodiments, x-rays 60 may be used in a medical application. In particular embodiments, using the x-rays in a medical application may comprise using the x-rays to produce an image or using the x-rays in medical radiotherapy and to produce an image. For example, referring to FIG. 8, an image detector 90 may be configured to produce an image.

In some embodiments, the medical application may comprise medical radiotherapy, cancer therapy, radiation oncology, tumor therapy, and/or activating a drug, amongst other. In embodiments where the medical application comprises activating a drug, activating the drug may comprise irradiating the drug. The drug may comprise a drug used for tumor therapy. The drug may act as a radiation sensitizer for tumor therapy. The drug may comprise a compound that contains a heavy element, for example, iodine, gadolinium, and platinum. The drug may comprise an lodinated Deoxyuridine (IUdR).

In some embodiments, a tumor 80 which is inside a body part 70 is dosed with a drug. Again, the drug may be any of the ones described above. Thus, the drug may be an IUdR, and as such the tumor may be an IUdR-dosed tumor 80 as shown in FIG. 7. Body part 70 may be that of a human or any other animal. IUdR-dosed tumor 80 is irradiated with x-rays 60 above the k-absorption edge of the heavy element of the drug. The drug may exhibit absorption, and after absorbing an x-ray photon may release the captured energy in an Auger cascade of electrons. The drug may bind to the DNA in the nucleus of a cell within tumor 80, and such a cascade may kill the cell. In some embodiments, effective radiation dose to tumor cells (as measured by cell death) may be 3-5 times higher than to cells around the tumor which have not taken up the drugs.

The x-rays 60 that may be generated by the EBIT may interact with cisplatin, IUdr, or any other heavy element drug and may generate Auger electrons at sites of drug incorporation within DNA. This combined effect of a DNA double strand break adjacent to site of incorporation of drug into DNA may enhance the interaction between drug and radiation as compared to the traditional approach of using megavoltage radiation. The EBIT irradiation may achieve greater biological effect in human cancer cell lines as compared to megavoltage irradiation.

All of the methods and systems disclosed and claimed can be made and executed without undue experimentation in light of the present disclosure. While the methods of this invention have been described in terms of embodiments, it will be apparent to those of skill in the art that variations may be applied to the methods and in the steps or in the sequence of steps of the method described herein without departing from the concept, spirit and scope of the invention.. All such similar substitutes and modifications apparent to those skilled in the art are deemed to be within the spirit, scope, and concept of the disclosure as defined by the appended claims.

### REFERENCES

Each of the following references is hereby incorporated by reference in its entirety:

Bernieretal., N. Engl. J Med., 350:1945-1952, 2004.

Biston et al., Cancer Res., 640:2317-2323, 2004.

Bloomer and Adelstein, Nature, 254(3393):620-621, 1977.

Bloomer and Adelstein, Pathobiol Annu., 8:407-421, 1978a.

Bloomer and Adelstein, Curr. Top Radiat. Res. Q, 12(1-4):513-525, 1978b.

Carroll et al., Amer. J Roetgenology, 191(5):f 197-1202, 2003.

Cooper et al., N. Engl. J Med., 350:1937-1944, 2004.

Corde et al., Brit. J Cancer, 91:544-551, 2004.

Debusk et al., Exp. Cell Research, 298(1):167-177, 2004.

Edwards et al., Cancer Research, 62:4671-4677, 2002.

Eichler, In: AIP Proceedings—The Physics of Electronic and Atomic Collisions, XXI Intl. Conference, 500:520-529, AIP, 2000.

Elliott, Nucl. Instru. Methods in Physics Res. Sec. B: Beam Interactions Mat. Atoms, 98(1-4):114-121, 1995.

Fairchild and Bond, Strahlentherapie, 160(12):758-763, 1984.

Feinendegen, Radiat. Environ. Biphys., 12(2):85-99, 1975.

Forastiere et al., N. Engl. J. Med., 349:2091-2098, 2003.

Geng et al., Cancer Res., 61(6):2413-2419, 2001.

Geng et al., Cancer Research, 64(14):4893-4899, 2004.

Gillaspy et al., Physica. Scripta, T59:392-395, 1995.

Hallahan et al., Intl. J Radiat. Oncol. Biol. Phys., 19:69-74, 1990.

Hallahan et al., Nature Medicine, 1:786-791, 1995.

Hallahan et al., Radiation Research, 129:345-350, 1992.

Hofer et al.,. Curr. Top Radiat. Res. Q, 12(1-4):335-354, 1978.

Jackson, In: Classical Electrodynamics, 2^{nd} Ed., Wiley Press, NY, 14(7):680-681, 1975.

Karnas et al, Phys. Med. Biol., 44:2537-2549, 1999.

Keough and Hofer, Radiat. Res., 67(2):224-234, 1976.

Laster et al., Acta Oncol, 35(7):917-923, 1996.

Levine et al., Phsica Scripta, T22:157, 1988.

Lu et al., Intl. J Radiation Oncology Biology Physics, 58:844-850, 2004.

Marrs, Nucl. Instru. Methods in Physics Res. Sec. B: Beam Interactions Mat. Atoms, 149(1-2):182-194, 1999.

Marrs et al., Physical Rev. Letters, 72:4082-4085, 1994.

Movsas et al., J. Clin. Oncol., 21(24):4553-4559, 2003.

Nath et al., Int. J Radiat. Oncol. Biol. Phys., 13(7):1071-1079, 1987.

Pikin et al., Rev. Scientific Instruments, 67(7):2528-2533, 1996.

Schuenemann et al., Cancer Research, 63:4009-4016, 2003.

Schulz et al., Intl. J Radiation Oncology Biology Physics, 59(4): 1107-1115, 2004.

Solberg et al., Phys. Med. Biol., 37(2):439-443, 1992.

Stolterfoht et al., In: AIP Proceedings: The physics of Electronic and Atomic Collisions, XXI Intl. Conference, 500:646-655, AIP, 2000.

Takacs et al., Nucl. Instru. Methods in Physics Res. Sec. B: Beam Interactions Mat. Atoms, 124(2-3):431-434, 1997.

Tan and Hallahan, Cancer Research, 63:7663-7667, 2003.

Waters, MCNPX User's Manual, Version 2.1.5, Technical Reports, Los Alamos Natl. Laboratory, 1999.

1. A method for using x-rays in a medical application, comprising:
extracting ions from an EBIT;
transporting the ions through an evacuated tube;
producing x-rays by neutralizing the ions when the ions strike a conducting plate; and
delivering the x-rays to an area of a body comprising a tumor.

2. The method of claim 1, where the ions comprise stripped ions.

3. The method of claim 1, where the ions comprise hydrogenic ions.

4. The method of claim 1, where transporting the ions through an evacuated tube comprises transporting the ions as a beam through an evacuated tube.

5. The method of claim 4, where the beam comprises a focusable beam.

6. The method of claim 4, where the beam comprises a steerable beam.

7. The method of claim 1, where the X-rays comprise non-continuous-spectrum x-rays.

8. The method of claim 1, further comprising filtering l-series energy photons from the x-rays.

9. The method of claim 1, further comprising selecting a variety of ions, where selecting a variety of ions comprises selecting an x-ray energy.

10. (canceled)

11. The method of claim 1, where delivering the x-rays comprises delivering the x-rays externally.

12. The method of claim 1, where delivering the x-rays comprises delivering the x-rays to a center of the tumor.

13. The method of claim 1, the tumor comprising a drug.

14. The method of claim 13, where the step of delivering the x-rays comprises activating the drug.

15. The method of claim 13, the drug comprising iodine, gadolinium, or platinum.

16. The method of claim 13, the drug comprising iododeoxyuridines (IUdRs) or cis-diamminedichloroplatinum (II) (cDDP).

17. A method for irradiating a tumor in a patient, comprising:
providing the tumor with a radiation sensitizer agent;
delivering x-rays to the patient, where the x-rays are produced by extracting ions from an EBIT;
transporting the ions through an evacuated tube;
neutralizing the ions when the ions strike a conducting plate to produce the x-rays;
activating the radiation sensitizer agent with the x-rays;
irradiating the tumor.

18. The method of claim 17, where the ions comprise stripped ions.

19. The method of claim 17, where the ions comprise hydrogenic ions.

20. The method of claim 17, the radiation sensitizer agent comprising iodine, gadolinium, or platinum.

21. The method of claim 17, where delivering the x-rays comprises delivering the x-rays externally.

22. The method of claim 17, where delivering the x-rays comprises delivering the x-rays to the center of the tumor.

23. A system for generating x-rays, comprising:
an EBIT configured to emit ions;
an evacuated tube configured to transport the ions;
a conducting plate configured to produce x-rays by neutralizing the ions when the ions strike the conducting plate;
a filter configured to filter l-series energy photons from the x-rays, where the x-rays are delivered to an area of a body comprising a tumor

24. The system of claim 23, the EBIT configured to emit stripped ions.

25. The system of claim 23, the EBIT configured to emit hydrogenic ions.

26. The system of claim 23, where the evacuated tube is configured to transport the ions as a beam.

27. The system of claim 26, where the beam comprises a focusable and steerable beam.

28. The system of claim 23, where the x-rays include essentially non continuous-spectrum x-rays.

29. The system of claim 23, further comprising a selector configured for selecting a variety of ions, where selecting a variety of ions comprises selecting an x-ray energy.

30. The system of claim 23, further comprising an image detector configured to produce an image.

31. The system of claim 23, where the x-rays comprise tunable X-rays.

32. The system of claim 31, where the tunable x-rays comprise step-tunable X-rays.

33. The system of claim 23, where the x-rays comprise line x-rays at a variety of discrete energies with sufficient intensity for a medical application.
